(19) United States
(12) Patent Application Publication
(10) Pub. No.: US 2021/0356066 A1
(43) Pub. Date: Nov. 18, 2021

(54) Pipeline-Inspecting Device

(71) Applicant: Dennis Frederick Viviers, Benoni (ZA)

(72) Inventor: Dennis Frederick Viviers, Benoni (ZA)

(21) Appl. No.: 16/321,226

(22) PCT Filed: Jul. 21, 2017
(86) PCT No.: PCT/ZA2017/050039

§ 371 Date: Jan. 28, 2019

(30) Foreign Application Priority Data
Jul. 27, 2016 (ZA) 2016/05227

(51) Int. Cl. F16L 55/40; G01N 27/02; G01N 33/2045; G01B 11/24; F16L 55/32

(52) U.S. Cl. CPC F16L 55/40; G01N 27/02; F16L 2101/30; G01B 11/2408; F16L 55/32; G01N 33/2045

(57) ABSTRACT

This invention relates to a pipeline-inspecting device. More specifically, the invention relates to a pipeline-inspecting device capable of at least (i) detecting defects in the lining of the pipeline; (ii) visually marking each of the defects in the pipeline; and (iii) recording the location of each of the defects along such pipeline in a single pass. The pipeline-inspecting device includes a primary body (20) being operably movable along a pipeline, fitted with a plurality of primary contacts (40) extending radially from the primary body (20) for operably riding in contact with an internal surface of the pipeline, the contacts (40) being spaced circumferentially relative to one another through 360 degrees about the primary body (20). The device further includes one or more secondary contacts (116) for operably connecting the device to the pipeline and a plurality of marking members (66) associated with each of the respective contacts (40) for visually marking the internal surface of the pipeline in the vicinity of defects detected therein, wherein the defects are operably detected by monitoring an electrical condition change between the primary (40) and secondary contacts (116). In use, and in the event of an electrical condition change arising between one of the primary contacts (40) and the secondary contact (116), the marking member associated with that respective primary contact (40) marks the internal surface of the pipeline in the vicinity of the defect detected by such primary contact (40).

### BACKGROUND OF THE INVENTION

THIS invention relates to a pipeline-inspecting device. More specifically, the invention relates to a pipeline-inspecting device capable of at least (i) detecting defects in the lining of the pipeline; (ii) visually marking each of the defects in the pipeline; and (iii) recording the location of each of the defects along such pipeline in a single pass.

Pipeline-inspecting devices are well known. A first of these prior art devices is commonly referred to the art as a wet sponge tester. This device (as well as the other prior art devices) operate by detecting defects through the monitoring of electrical conductivity between an earth wire, connected to a bare metal section of the pipeline, and a single wetted sponge contactor that is wiped across the internal lining of the pipeline manually by an inspector.

On passing the wetted sponge contactor over a defect in the lining of the pipeline, an electrical circuit is completed between the earth wire and the wetted sponge contactor providing the inspector with a defect alert. The manual operation of this device makes inspection very time consuming, cumbersome, expensive and inaccurate.

A second of these prior art devices is commonly referred to as a pipeline crawler or pig, which are remotely controller and outfitted with cameras and/or probes. The devices fitted with cameras typically provide a recorded or real-time visual of the lining of the pipeline, whereas the devices fitted with probes (i.e. sonar probes) measure the thickness of pipeline wall. These devices generally do not incorporate the capability of visually marking the defect and/or recording its location. Also, these devices, particularly those fitted with probes, are expensive and sensitive to being mishandled.

A third of these prior art devices is taught by Langley, et al. in U.S. Pat. No. 7,077,020 disclosing an internal field joint inspection robot having mounted rotatable thereon a copper brush contact adapted to sweep the against the lining of the pipeline as the robot progresses longitudinally along the pipeline, i.e. such that the copper brush contact follow a spiral path along the pipeline. On detection of a defect, electrical conductivity is established between the brush contact and an earth wire connected between the robot and the pipeline, causing a spark and triggering the visual marking, location recording and video recording of the defect.

Although these robots overcome many of the disadvantages of the other known prior art devices, they too suffer from certain shortcomings, with the most concerning one being that the brush contact is prone to rapid wear, requiring frequent costly replacement and unnecessary downtime.

It is an object of the present invention to provide a pipeline-inspecting device that addresses the shortcomings of the known prior art devices.

### SUMMARY OF THE INVENTION

According to the invention there is provided a pipeline-inspecting device including:
a primary body being operably movable along a pipeline;
a plurality of primary contacts extending radially from the primary body for operably riding in contact with an internal surface of the pipeline, the contacts being spaced circumferentially relative to one another through 360 degrees about the primary body;
one or more secondary contacts for operably connecting the device to the pipeline; and
a plurality of marking members associated with each of the respective contacts for visually marking the internal surface of the pipeline in the vicinity of defects detected therein, wherein the defects are operably detected by monitoring an electrical condition change between the primary and secondary contacts;
such that in the event of an electrical condition change arising between one of the primary contacts and the secondary contact, the marking member associated with that respective primary contact marks the internal surface of the pipeline in the vicinity of the defect detected by such primary contact.

Generally, the primary body has a central axis about which the primary contacts are circumferentially spaced relative to one another, wherein in use, the central axis of the primary body is parallel to and/or co-axial with a central longitudinal axis of the pipeline.

Typically, lateral ends of adjacent primary contacts lie in abutment or close proximity to one another in a circumferential side-by-side relationship. In this manner, the cumulative contact dimension of the primary contacts with the internal surface of the pipeline is substantially the same as the circumference of such internal surface.

Alternatively, and with reference to the central axis of the primary body, the primary contacts lie in a staggered configuration relative to one another such that adjacent primary contacts are: (i) axially offset relative to one another; and (ii) comprise circumferentially overlapping lateral ends. In this manner, the cumulative contact dimension of the primary contacts with the internal surface of the pipeline is greater than the circumference of such internal surface.

Preferably, the primary contacts are mounted to the primary body by respective support arms on which the primary contacts are movable in radially inward and outward directions relative to the central axis of the primary body, with the primary contacts being biased towards the radially outward direction.

The support arms may have opposing first and second ends and may each be hingedly connected to the primary body at a respective pivot located between such first and second ends. Furthermore, the primary contact mounted on each of the support arms may be located nearer the first end thereof with a biasing means, for biasing the primary contacts in the radially outward direction, acting on such support arms between the second end thereof and the respective pivot.

Generally, the biasing means are springs connected between the support arms and (i) the primary body; or (ii) a tension support member mounted on the primary body. Alternatively, the biasing means are one or more elastic bands captured jointly by hook-like formations at or near second ends of the support arms.

Typically, each of the primary contacts is a contact sponge, mounted on the first end of the respective support arms by a clamping member, such that a contacting portion of the sponge extends outwardly beyond a first edge of the clamping member.

A spacing element may be mounted on each of the support arms between its respective pivot and the contact sponge thereof, with the spacing elements being configured for operably riding along the internal surface of the pipeline thereby to maintain a substantially constant distance between the internal surface of the pipeline and the first edge of the clamping member.

Preferably, the spacing elements are spacer wheels and the marking members are marking nozzles for spraying a marking fluid. More preferably, the primary body is movably supported along the pipeline on a plurality of support wheels.

In a particularly preferred embodiment, the device may also include wetting nozzles for wetting the contact sponges and/or the internal surface of the pipeline with water or another electrically conductive fluid, wherein the marking fluid and the water are stowable in respective pressurised holding tanks.

Generally, the secondary contact is a long wire operably connected between the device and a bare metal section of the pipeline.

Typically, the device includes any one or more of:
a means of tracking the location of the device along the pipeline thereby to operably record the positions of the defects detected;
one or more cameras for capturing photographs and/or video of the defects operably detected;
a laser for: detecting out-of-roundness of the pipeline; detecting dents in the pipeline; and/or mapping the pipeline;
a drive means for driving the device along the pipeline;
a power supply for supplying power to the components of the device requiring power;
a controller for controlling the operations of the device;
memory for storing operational software, and/or (i) tracking data from the tracking means; and/or (ii) photograph and/or video data from the cameras; and
a communications means for enabling communications between the device and an operator inspection device, the communications means enabling transmission therebetween of one or more of:
(i) tracking data;
(ii) laser data;
(iii) photograph and/or video data from the memory or live in real-time; and
(iv) control signals for controlling at least the drive means.

Preferably, the tracking means is: a global positioning system (GPS); a means for measuring the amount of wire or cable reeled in or out from a spool mounted on a spool station, such that the relative distance between the device and the spool station along the pipeline can be monitored; or a combination of the global positioning system and the spool measuring means.

The primary body may be annular and movable along the pipeline substantially concentrically therealong. Furthermore, the support wheels may be castors and/or suspension mounted to the primary body thereby to accommodate cross-sectional inconsistencies in the pipeline.

Generally, the device comprises the primary body and a secondary annular body for towing the primary body, the secondary body being operably movable along the pipeline on a plurality of suspension mounted drive wheels, wherein:
(i) the tracking means is carried on the secondary body and/or on the spool station;
(ii) the drive means, the controller and at least parts of the power supply and the communications means are mounted on the secondary body;
(iii) the cameras and laser are mounted on the primary body together with the primary contacts, the support arms and the biasing means; and
(iv) the holding tanks are mounted on either of the primary and/or the secondary bodies.

Typically, the secondary body comprises a central axis being operably parallel and/or co-axial with the central axis of the primary body, with the primary and the secondary bodies being connected via a flexible joint. It will be appreciated that the flexible joint enables the device to operably navigate through bends in the pipeline without becoming lodged therein.

Preferably, a stepper motor drives each of the drive wheels.

The wheels of the primary and the secondary bodies may be two or more such wheels on each of the primary and the secondary bodies, wherein the wheels on each of such bodies is angularly spaced relative to one another about the respective central axis by about 120 degrees.

Generally, the flexible joint connecting the primary and the secondary bodies to one another is supported by a plurality of connecting arms extending from each of the primary and the secondary bodies such that the flexible joint lies substantially on or near the central axes of the primary and the secondary bodies.

Typically, the cameras and/or laser are mounted on the primary body: by a plurality of mounting arms extending from the primary body; or within a cylindrical housing having at least one transparent portion; such that the cameras are located near the central axis of the primary body, on a side thereof being opposite a side on which the flexible joint is located relative to the primary body.

The spool may be mounted on the device or on the spool station.

According to a second aspect of the invention there is provided a method of inspecting a pipeline in a single pass including the steps of:
(A) inserting a pipeline-inspecting device into pipeline to be inspected such that a plurality of circumferentially spaced primary contacts extending from the pipeline-inspecting device remains in contact with a 360 degree band of an internal surface of the pipeline
(B) electrically connecting a second contact of the pipeline-inspecting device to the pipeline;
(C) monitoring any changes in voltage, resistance, impedance, conductivity or other electrical property between the primary contacts and the pipeline as the pipeline-inspecting device moves along the pipeline;
(D) on detecting a change in electrical property between the primary contact and the pipeline as a result of a defect, actuating a marking device associated with such primary contact to visually mark the internal surface of the pipeline in the vicinity of the defect detected; and
(E) recording the location of the defect.

### BRIEF DESCRIPTION OF THE INVENTION

The invention will now be described in more detail, by way of example only, with reference to the accompanying illustrations, in which:

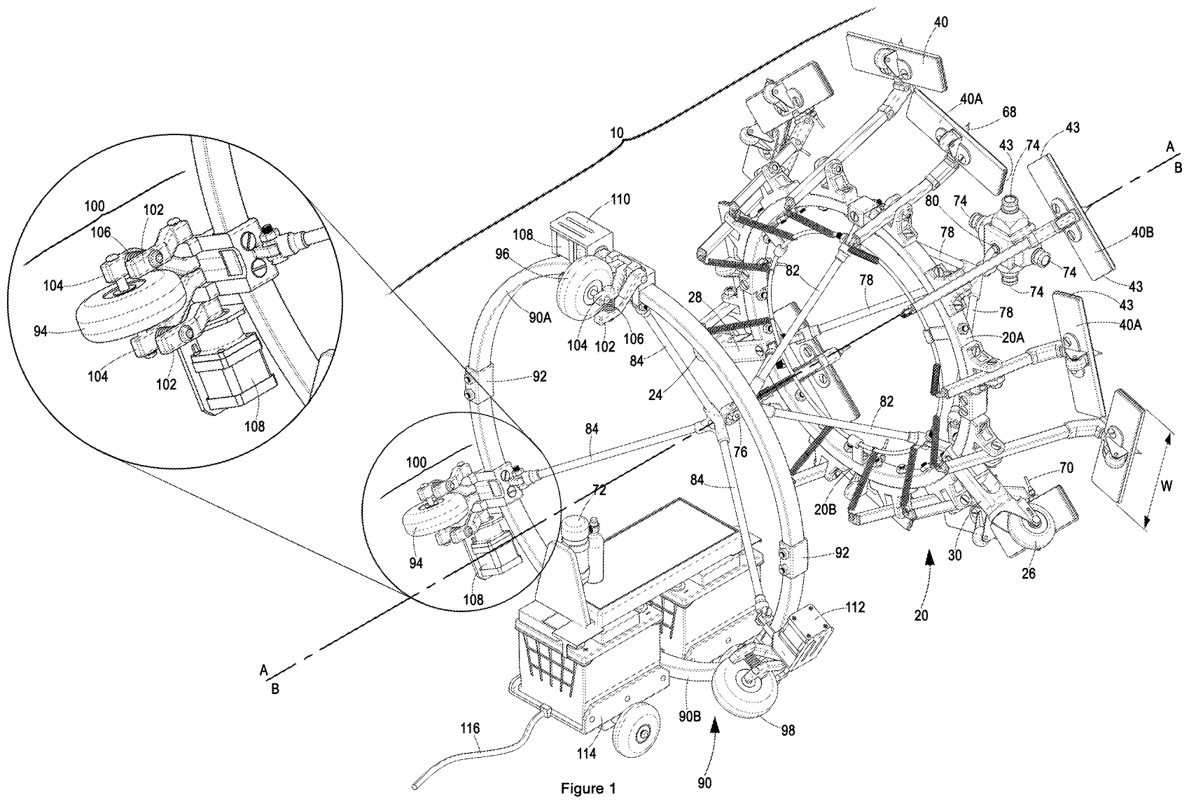

FIG. 1 is a perspective view of pipeline-inspecting device in accordance with the present invention, showing an enlarged view of a drive wheel of a secondary body of the pipeline-inspecting device;

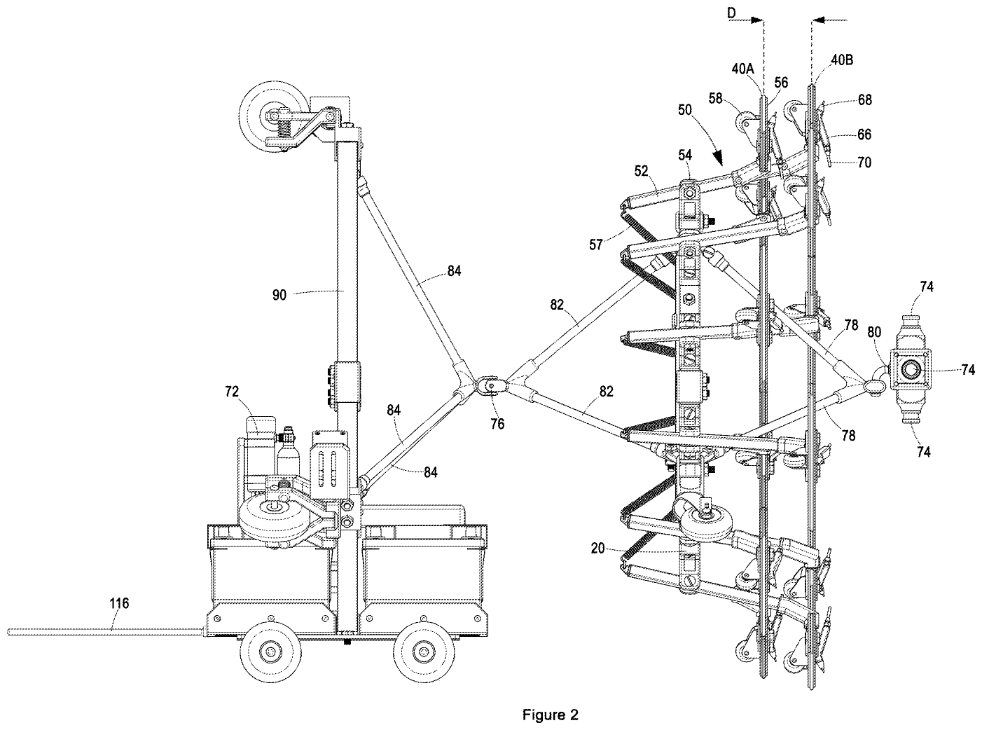

FIG. 2 is a side view of the pipeline-inspecting device of FIG. 1 in accordance with the present invention;

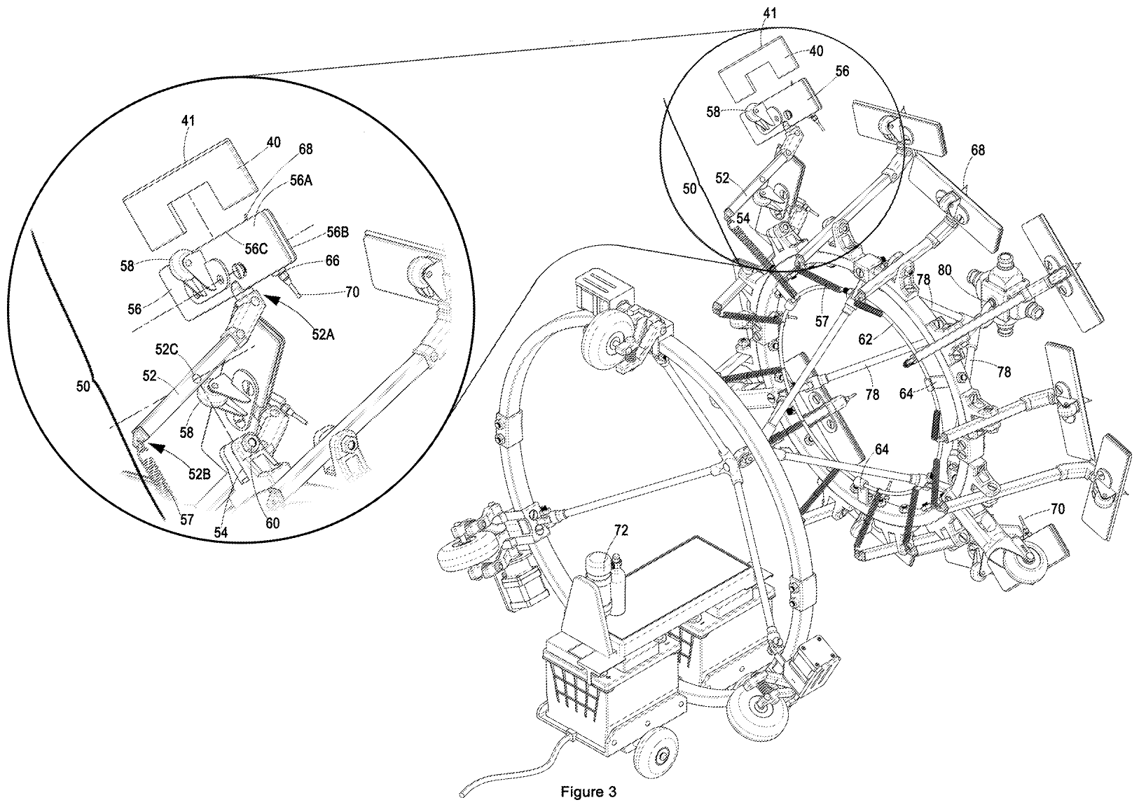

FIG. 3 is another perspective view of the pipeline-inspecting device, showing an enlarged view of a primary contact and a primary contact mounting sub-assembly for mounting the primary contact to a primary body of the pipeline-inspecting device;

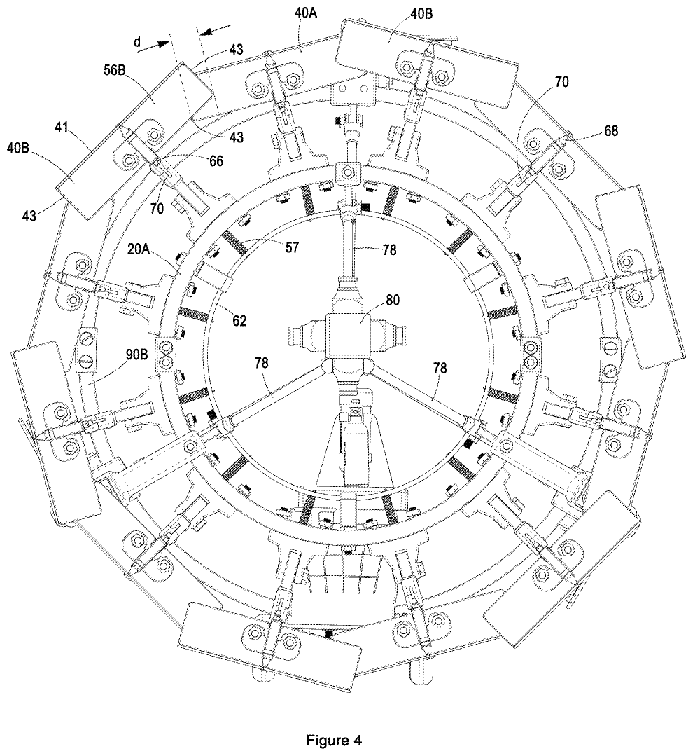

FIG. 4 is an end view of the pipeline-inspecting device of FIG. 1; and

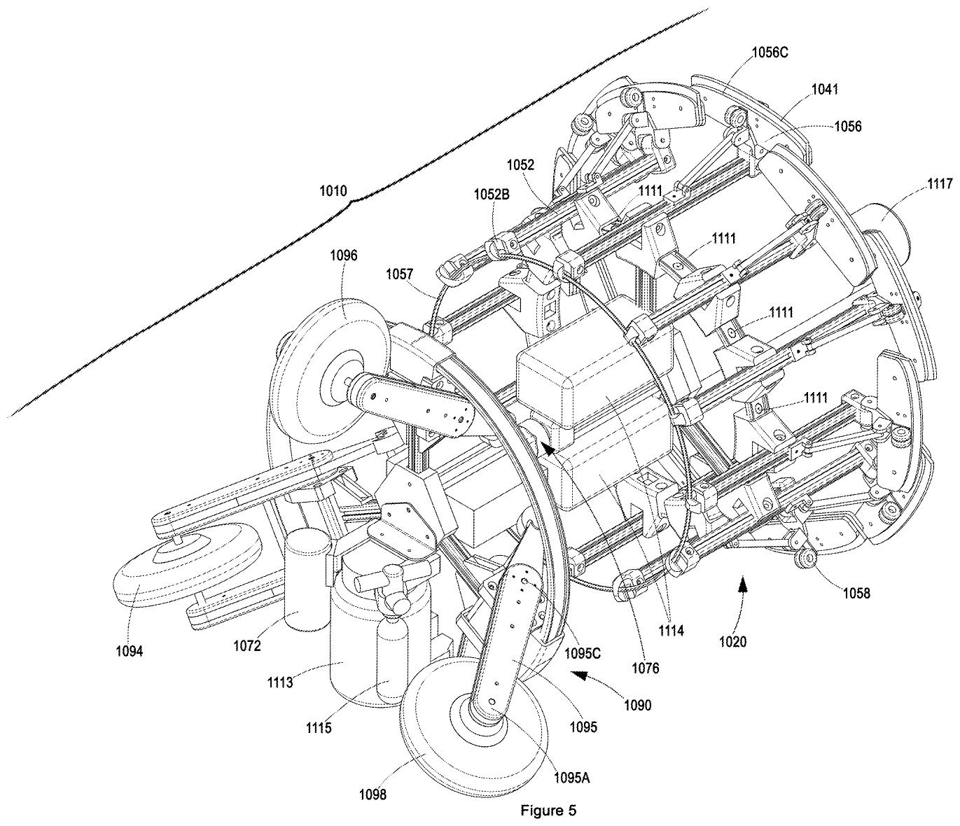

FIG. 5 is a perspective view of an alternative embodiment of a pipeline-inspecting device in accordance with the present invention;

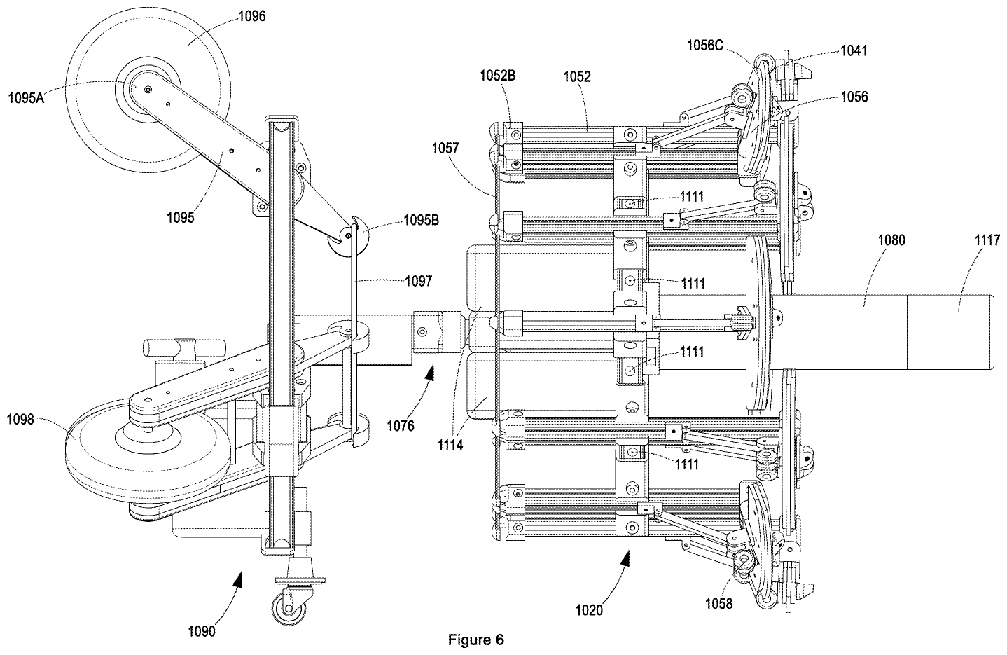

FIG. 6 is a side view of the pipeline-inspecting device of FIG. 5; and\

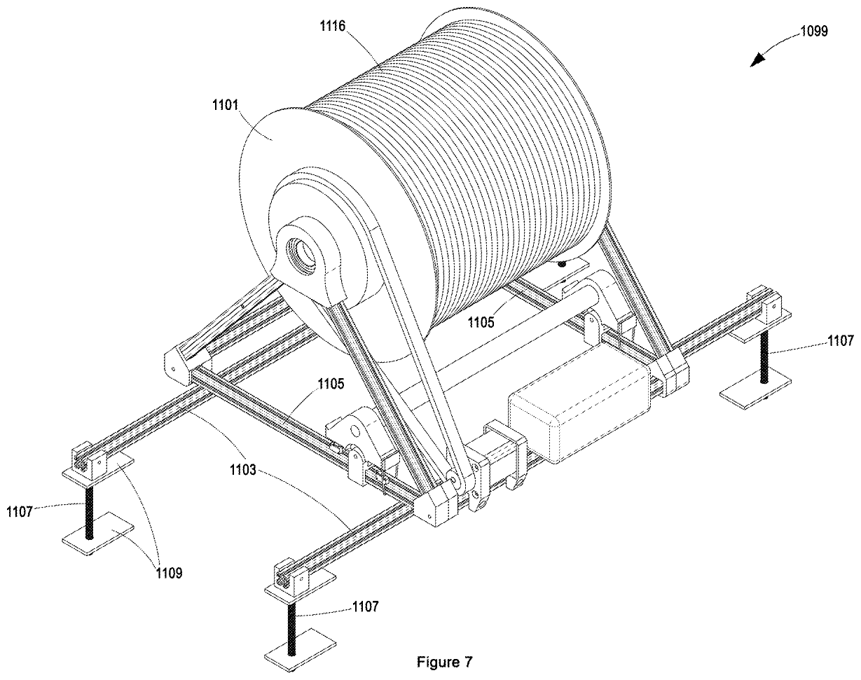

FIG. 7 is a perspective view of a spool station operatively for use together with the pipeline-inspecting device.

### DETAILED DESCRIPTION OF THE INVENTION

A pipeline-inspecting device according to a first embodiment of the invention is designated generally in the accompanying figures by reference numeral 10.

It will be appreciated that although it may be possible to incorporate all of the components making up the pipeline-inspecting device 10 into a single body, it is preferable that such components are split up between at least two articulated primary and second bodies 20, 90 for enabling the device 10 to operably navigate through a pipeline, particularly bends in the pipeline, without becoming lodged therein.

The primary body 20 is preferably annular in shape, having a central axis A-A passing therethrough, which central axis A-A is in use parallel to, or preferably co-axial with, a longitudinal central axis of the pipeline being inspected such that the pipeline-inspecting device 10 is movable through the pipeline substantially concentrically therewith.

The primary body 20 is made up of at least a pair of semi-circular primary body half sections 20A, 20B fixed together by diametrically opposing brackets 22. Furthermore, the primary body 20 is movable through the pipeline on at least two support wheels 24, 26 mounted on terminal ends of wheel support arms 28, 30 extending radially from the primary body 20 and being angularly spaced from one another by about 120 degrees. The support wheels support wheels 24, 26 may be castors and/or suspension mounted to the wheel support arms 28, 30.

The primary body 20 supports a plurality of circumferentially spaced primary contacts 40, with each of the primary contacts 40 hingedly mounted to the primary body 20 by a respective primary contact mounting sub-assembly 50.

With reference now also specifically to FIG. 3, each of the primary contact mounting sub-assemblies 50 comprises a support arm 52, a hinge bracket 54, a clamping member 56, a biasing member 57 and a spacing element in the form of a spacer wheel 58.

The support arm 52 comprises opposing first and second ends 52A, 52B, and defines a pivot hole 52C therebetween for receiving a fastener 60 therethrough to hingedly secure the support arm 52 to the hinge bracket 54 such that the primary contact 40 mounted thereon is moveable, with respect to the central axis A-A, in radially inward and outward directions.

The primary contact 40, typically in the form of a contact sponge, is mounted on the first end 52A of the support arm 52 by the clamping member 56. The clamping member 56 is made up of first and second clamping plates 56A, 56B between which the contact sponge 40 is securable, such that a contacting portion 41 of the contact sponge 40 extends outwardly beyond a first edge 56C of the clamping member 56 thereby to operably come into contact with an internal surface of the pipeline being inspected, more particularly a non-electrical conducting internal lining.

The biasing means 57, typically in the form of a spring, is connected at one end to the second end 52B of the support arm 52, and at an opposite end to some or other point on the primary body 20, but generally to an annular tension support member 62 mounted on the primary body 20 by a plurality of tension support brackets 64. It will be appreciated that in this manner, with the support arm 52 acting as a spring biased lever, the contact sponge 40 is biased towards the radially outward direction.

The spacer wheel 58 may be mounted at any location near the first end 52A of the support arm 52, but preferably on the clamping member 56. The purpose of the spacer wheel 58 is to maintain a substantially constant distance between the internal surface of the pipeline and the first edge 56C of the clamping member 56 thereby to reduce the force acting on the contacting portion 41 of the contact sponge 40 that would normally be exerted without such spacer wheel 58.

In this manner, the plurality of primary contacts 40 extend radially from the primary body 20, spaced circumferentially relative to one another through 360 degrees about such primary body 20.

With reference to the central axis A-A of the primary body 20, and as more clearly illustrated in FIG. 2 and FIG. 4, the primary contacts 40 lie in a staggered configuration relative to one another such that adjacent primary contacts 40A, 40B are axially offset relative to one another by a distance “D”, and have lateral ends 43 that circumferentially overlapping one another by a distance “d” such that the cumulative contact dimension (width “W” of the contacting portion 41 of the contact sponge 40 multiplied by the number of contact sponges 40) is greater than the circumference of the internal surface of the pipeline.

To the clamping member 56 is mounted a marking member 66, in the form of a sprayer having a marking nozzle 68 for dispensing a marking fluid therefrom onto the internal surface of the pipeline, and a marking fluid inlet 70 for delivering through a feed tube (not shown) marking fluid contained in a pressurised marking fluid holding tank 72 on the secondary body 90.

Although also not shown, the pipeline-inspecting device 10 is preferably fitted with a second set of water sprayers and a pressurised water holding tank for in use wetting the contact sponges 40 and/or the internal surface of the pipeline, thereby to increase the electrical conductivity of the primary contacts 40.

The primary body 20 has further mounted thereto a plurality of cameras 74 and a universal joint 76, the cameras 74 and the universal joint 76 being mounted on opposing first and second axial sides of the primary body 20.

The cameras 74 are mounted to the primary body 20 by three camera mounting arms 78 extending between the primary body 20 and a camera mounting bracket 80, such that the cameras 74 are located near the central axis A-A of the primary body 20 and directed in different radial directions relative to one another.

Similarly, the universal joint 76 is mounted to and between each of the primary and the secondary bodies 20, 90 by respective sets of three universal mounting arms 82, 84 extending between the primary and the secondary bodies 20, 90 and the universal joint 76. In this manner, the universal joint 76 is located near the central axis A-A of the primary body 20, and consequentially near a central axis B-B of the secondary body 90 being substantially parallel with and preferably co-axial with the central axis A-A of the primary body 20.

The secondary body 90 is preferably also annular in shape and made up of at least a pair of semi-circular secondary body half sections 90A, 90B fixed together by diametrically opposing brackets 92. In use, the secondary body 90 tows the primary body 20 such that they both travel substantially concentrically through the pipeline.

The secondary body 90 is movable through the pipeline, and preferably drives the pipeline-inspecting device 10 therethrough, on three radially outwardly extending drive wheels 94, 96, 98, each being suspension mounted to the secondary body 90 by respective suspension brackets 100, made up of a plurality of suspension arms 102, 104 with a suspension spring 106 acting therebetween.

It will be appreciated that the suspension mounting of the drive wheels 94, 96, 98 enables the secondary body 90 to accommodate cross-sectional inconsistencies in the pipeline. It will be appreciated further that by drive wheels 94, 96, 98 are angularly spaced from one another about the central axis B-B of the secondary body 90 by about 120 degrees to provide the secondary body 90 with stability.

Although the drive wheels could be driven by a single drive means, it is preferable that each drive wheel is driven by an independent drive means 108, 110, 112 in the form of stepper motors.

The secondary body 90 includes a wheeled housing 114 thereon for housing one or more of a means of tracking the location of the device 10, a power supply, a controller, memory, a communications means and associated electronics.

The tracking means is capable in use of recording the positions along the pipeline at which defects are detected. The tracking means may be a reference beacon type tracking system, wherein the distances between the device 10 and one or more static reference beacons are recorded, or preferably a global positioning system (GPS).

The power supply may be one or more batteries for powering the components of the device 10 requiring power, i.e. the drive means, the tracking means, the controller, the memory, the communications means, the marking members, water sprayers and the cameras.

The controller controls the operations of the device 10 through software inputs and/or operator control signals transmittable to the device from an operator inspection device (not shown), i.e. an electronic tablet.

The communications means enables the transmission of the operator control signals, as well as the transmission of tracking data and photograph and/or video data captured by the cameras. It will be appreciated that the tracking data and the photograph and/or video data may be stored on the memory of the device 10, and/or live streamed in real-time to the operator inspection device.

It will be appreciated that on a defect being detected by the device 10, a defect record will be created associating together any two or more of the following: (i) the position of the defect as recorded by the tracking means; (ii) a visual of the defect and/or a physical marking in the vicinity of the defect (to be described later in this description with reference to an alternative embodiment of the invention) as captured by the cameras; (iii) an identifier of the primary contact 40 having detected the defect for the purposes of recording a circumferential position of the defect at the relevant axial position along the pipeline and/or GPS co-ordinate; (iv) the date and time of the inspection; and (v) the approximate size of the defect as calculated from a electrical condition change caused by such defect.

The pipeline-inspecting device 10 further comprises a secondary contact 116, typically in the form of a long wire extending from the pipeline-inspecting device 10 and being connectable at an opposite end to a bare metal section of the pipeline, thereby to electrically connect or earth the pipeline to the pipeline-inspecting device 10, more particularly to the primary contacts 40, such that an electrical detection circuit is created by the primary contacts 40 riding along the internal lining of the pipeline, the pipeline itself and the secondary contact wire 116.

The pipeline-inspecting device 10 may further comprise a spool (not shown) onto or from which the secondary contact wire 116 may be spooled as the device moves through the pipeline. The spool may be located statically at some point along the pipeline, or mounted on the device 10.

In use, the internal lining of the pipeline acts as an isolator to break the electrical detection circuit whilst the pipeline-inspecting device 10 moves through the pipeline. As a respective wetted primary contact 40 passes over a defect in the internal lining, and consequentially over an area of the pipeline exposed by such defect, the defect-exposed area closes the electrical detection circuit. Accordingly, the defect-exposed area acts as a switch to establish electrical conductivity between the primary contacts 40, the pipeline and the secondary contact wire 116.

It will be appreciated then that the defects are operably detected by monitoring an electrical condition change as measured between the primary and secondary contacts 40. It will be appreciated further that this change in electrical condition may be used to trigger the operation of the marking members 66 in a number of different ways.

For example, the controller may be configured to monitor a voltage, resistance, impedance, conductivity or other electrical property, and on detection of a change therein, triggering an output signal to the marking member 66 associated with the wetted primary contact 40 having detected the defect to mark the internal lining in the vicinity of the defect detected.

Alternatively, each of the primary contacts 40 have independent circuits connected to its associated marking member 66 such that on the wetted primary contact 40 passing over a defect, the respective independent circuit is energise thereby to power the marking member 66 to mark the internal lining in the vicinity of the defect detected.

In this manner, the positions of the defects are visually marked on the internal lining along the pipeline, with such positions and visuals captured by the cameras capable of being associated with each and every defect detected.

Not only does the pipeline-inspecting device 10 provide a quicker and more accurate means of inspecting a pipeline as compared to the known prior art devices, but also a means for providing a client with an accurate account of the number of defects detected, which is typically how the full inspection cost is calculated and invoiced (i.e. price per defect detected multiplied by the actual number of defects detected).

Although the invention has been described with reference to a preferred embodiment, it will be appreciated that many modifications or variations of the invention are possible without departing from the spirit or scope of the invention. For example, instead of:
adjacent primary contacts 40 being staggered, they may be configured to lie in abutment or close proximity to one another in a circumferential side-by-side relationship;
the primary contacts 40 being hingedly supported on the primary body 20 by the support arms 52, the supporting arms may be telescopic and movable linearly in a radial direction; and
the marking members 66 being configured to spray a marking fluid in the form of paint or ink as envisaged by the inventor, the marking members 66 may be pen-like markers that move between extended and retracted conditions so as to come into contact with the internal lining in the extended condition.

FIG. 5 and FIG. 6 illustrate an alternative embodiment of the pipeline-inspecting device 1010, with like references designating like components. The description that follows focuses on the main differences between the first embodiment of the pipeline-inspecting device 10 and this alternative embodiment 1010.

The pipeline-inspecting device 1010 similarly comprises of two articulated primary and second bodies 1020, 1090, where the connection 1076 therebetween may be a universal joint, a ball joint, a flexible connector or the like. The primary body 1020 of this pipeline-inspecting device 1010 relies on the spacer wheels 1058 to not only maintain a substantially constant distance between the internal surface of the pipeline and the first edge 1056C of the clamping member 1056, but also to support the primary body 1020 within the pipeline through the tension applied onto the support arm 1052 by the biasing member 1057. In this manner support wheels 24, 26, as forming part of the first embodiment 10 of the invention, may be done away with.

The pipeline-inspecting device 1010, instead of making use of springs as the preferred biasing means 1057, makes use of an elastic band captured jointly by hook-like formations on the second ends 1052B of each of the support arms 1052. An elastic band type biasing means 1057 provides a quick and cost effective means of tuning into the contact force required between the contacting portion 1041 and the pipeline. A simple replacement of the elastic band 1057, with another of a different tension, enables the contact force to be adjusted efficiently.

Through testing, a better method of configuring the drive wheels 1094, 1096, 1098 of the secondary body 1090 has been uncovered. In the alternative embodiment 1010, the drive wheels 1094, 1096, 1098 are connected at first ends 1095A of lever arms 1095, pivotally mounted to the secondary body 1090 at a pivot 1095C, which pivot 1095C lies intermediate the first end 1095A and a second end 1095B of the respective lever arm 1095.

An elastic band 1097 is captured jointly by hook-like formations on the second ends 1095B of each of the lever arms 1095 thereby to splay the drive wheels 1094, 1096, 1098 radially outwardly. It will be appreciated that the simple replacement of the elastic band 1097 with one of a different tension will enable the contact force required between the drive wheels 1094, 1096, 1098 and the pipeline to be quickly tuned.

Although this alternative embodiment of the pipeline-inspecting device 1010 includes a housing 1114, the position of such housing 1114 has moved from near the operative bottom side of the secondary body 1090 to a location nearer the central axis A-A and between the primary and secondary bodies 1020, 1090.

The housing 1114 has been adapted to carry less or smaller batteries on-board, with the reduced on-board power being supplemented by an external power supply connectable to the housing 1114 by a contact wire (not shown), typically in the form of a cable including therein a fibre optic strand and multiple copper wire strands.

In use, the fibre optic strand communicates information (i.e. at least operator control inputs, camera feed, video feed, the axial position of the defect along the pipeline, the circumferential position of the defect circumferentially about the pipeline, the approximate size of the defect and laser detected outputs) between the pipelline-inspecting device 1010 and the operator inspection device.

The multiple copper wire strands will comprise at least positive and negative wires to power the pipelline-inspecting device 1010 as well as an earth connectible to the pipeline thereby to form the electrical detection circuit.

With reference now to FIG. 7, it has been found that the device 1010 works much more accurately by locating the tracking means on a spool station 1099 on which the spool 1101 for reeling in and/or out the cable 1116 is mounted. By measuring the amount of cable 1116 reeled in or out of the spool 1101 (spool measuring means), the axial position of the pipelline-inspecting device 1010 can be calculated. When coupled with a GPS tracking means, recording the co-ordinate of the spool station, the axial and circumferential positions of each defect can be calculated as a function of the GPS co-ordinate of the spool station 1099 and the amount of cable 1116 reeled on or off the spool.

The spool station 1099 is preferably supported on pairs of slides 1103, 1105 angulaly displaced relative to one another by 90 degrees thereby to enable an operator to slidably centre the spool station 1099 relative to an opening into the pipeline. At least one pair of the slides 1103 is supportable on legs 1107 having clamping formations 1109 thereon for clamping the spool station 1099 to the pipeline over the opening.

Although not shown on the first embodiment of the pipeline-inspecting device 10, the alternative embodiment 1010 thereof now shows the preferred position of the second set of water sprayers or misters 1111 and the pressurised water holding tank 1113.

The misters 1111 are each circumferentially spaced relative to one another about the primary body 1020, with the water holding tank 1113 located on the secondary body 1090. To pressurise the water holding tank 1113, the secondary body 1090 may support a pressurising means 1115, in the form a CO2 cylinder or compressor.

It will be appreicate that the marking fluid holding tank 1072 will also be pressurised by the pressursing means 1115, with the fluid being directable from the holding tank 1072 to a valve bank (not shown), with the valves of such valve bank being actuated to deliver fluid to the required marking member.

Referring no back to FIGS. 5 and 6, the alternative embodiment of the pipeline-inspecting device 1010 now also offers better protection to the cameras (not shown) housed within a transparent portion 1117 on the end of a camera housing 1080. It is envisaged that a laser capable of spinning about the central axis A-A (or an axis substantially parallel therewith) will be housed together with the cameras in the camera housing 1080. The laser will be used for to measure out-of-roundness of the pipe and dents, as well as for mapping out the pipeline.

It will be appreciated that any reference in this specification to the term defect will be understood to mean any opening in the internal lining, however small, through which a bare metal portion of the pipeline is exposed including holidays, pinholes, cracks and thin DFT's (Dry Film Thickness).

1. A pipeline-inspecting device including:
a primary body being operably movable along a pipeline;
a plurality of primary contacts extending radially from the primary body for operably riding in contact with an internal surface of the pipeline, the contacts being spaced circumferentially relative to one another through 360 degrees about the primary body;
one or more secondary contacts for operably connecting the device to the pipeline; and
a plurality of marking members associated with each of the respective contacts for visually marking the internal surface of the pipeline in the vicinity of defects detected therein, wherein the defects are operably detected by monitoring an electrical condition change between the primary and secondary contacts;
such that in the event of an electrical condition change arising between one of the primary contacts and the secondary contact, the marking member associated with that respective primary contact marks the internal surface of the pipeline in the vicinity of the defect detected by such primary contact.

2. A device according to claim 1, wherein the primary body has a central axis about which the primary contacts are circumferentially spaced relative to one another, wherein in use, the central axis of the primary body is parallel to and/or co-axial with a central longitudinal axis of the pipeline.

3. A device according to claim 2, wherein:
(i) lateral ends of adjacent primary contacts lie in abutment or close proximity to one another in a circumferential side-by-side relationship; or
(ii) and with reference to the central axis of the primary body, the primary contacts lie in a staggered configuration relative to one another such that adjacent primary contacts are: (i) axially offset relative to one another; and (ii) comprise circumferentially overlapping lateral ends.

4. A device according to claim 3, wherein the primary contacts are mounted to the primary body by respective support arms on which the primary contacts are movable in radially inward and outward directions relative to the central axis of the primary body, with the primary contacts being biased towards the radially outward direction.

5. A device according to claim 4, wherein the support arms have opposing first and second ends and are each hingedly connected to the primary body at a respective pivot located between such first and second ends, the primary contact mounted on each of the support arms being located nearer the first end thereof with a biasing means, for biasing the primary contacts in the radially outward direction, acting on such support arms between the second end thereof and the respective pivot.

6. A device according to claim 5, wherein the biasing means are:
springs connected between the support arms and: (i) the primary body; or (ii) a tension support member mounted on the primary body; or
one or more elastic bands captured jointly by hook-like formations at or near second ends of the support arms.

7. A device according to claim 6, wherein each of the primary contacts is a contact sponge, mounted on the first end of the respective support arms by a clamping member, such that a contacting portion of the sponge extends outwardly beyond a first edge of the clamping member.

8. A device according to claim 7 comprising a spacing element mounted on each of the support arms between its respective pivot and the contact sponge thereof, the spacing elements being configured for operably riding along the internal surface of the pipeline thereby to maintain a substantially constant distance between the internal surface of the pipeline and the first edge of the clamping member.

9. A device according to claim 8, wherein the spacing elements are spacer wheels and the marking members are marking nozzles for spraying a marking fluid.

10. A device according to claim 9, wherein the primary body is movably supported along the pipeline on a plurality of support wheels, and further wherein the support wheels are castors and/or suspension mounted to the primary body thereby to accommodate cross-sectional inconsistencies in the pipeline.

11. A device according to claim 10 including wetting nozzles for wetting the contact sponges and/or the internal surface of the pipeline with water or another electrically conductive fluid, wherein the marking fluid and the water are stowable in respective pressurised holding tanks.

12. A device according to claim 11, wherein the secondary contact is a long wire or cable operably connected between the device and a bare metal section of the pipeline.

13. A device according to claim 12 including any one or more of:
a means of tracking the location of the device along the pipeline thereby to operably record the positions of the defects detected;
one or more cameras for capturing photographs and/or video of the defects operably detected;
a laser for: detecting out-of-roundness of the pipeline; detecting dents in the pipeline;
and/or mapping the pipeline;
a drive means for driving the device along the pipeline;
a power supply for supplying power to the components of the device requiring power;
a controller for controlling the operations of the device;
memory for storing operational software, and/or (i) tracking data from the tracking means; and/or (ii) photograph and/or video data from the cameras; and
a communications means for enabling communications between the device and an operator inspection device, the communications means enabling transmission therebetween of one or more of:
(i) tracking data;
(ii) photograph and/or video data from the memory or live in real-time;
(iii) laser data; and
(iv) control signals for controlling at least the drive means.

14. A device according to claim 13, wherein the tracking means is:
a global positioning system (GPS);
a means for measuring the amount of wire or cable reeled in or out from a spool mounted on a spool station, such that the relative distance between the device and the spool station along the pipeline can be monitored; or
a combination of the global positioning system and the spool measuring means.

15. A device according to claim 14, wherein the primary body is annular and movable along the pipeline substantially concentrically therealong, the device further including a secondary annular body for towing the primary body, the secondary body being operably movable along the pipeline on a plurality of suspension mounted drive wheels, wherein:
(i) the tracking means is carried on the secondary body and/or on the spool station;
(ii) the drive means, the controller and at least parts of the power supply and the communications means are mounted on the secondary body;
(iii) the cameras and laser are mounted on the primary body together with the primary contacts, the support arms and the biasing means; and
(iv) the holding tanks are mounted on either of the primary and/or the secondary bodies.

16. A device according to claim 15, wherein the secondary body comprises a central axis being operably parallel and/or co-axial with the central axis of the primary body, the primary and the secondary bodies being connected via a flexible joint for enabling the device to operably navigate through bends in the pipeline without becoming lodged therein.

17. A device according to claim 16, wherein a stepper motor drives each of the drive wheels.

18. A device according to claim 17, wherein the drive wheels of the secondary body are two or more such wheels angularly spaced relative to one another about the central axis by about 120 degrees.

19. A device according to claim 18, wherein the cameras and/or laser are mounted on the primary body:
by a plurality of mounting arms extending from the primary body; or
within a cylindrical housing having at least one transparent portion;
such that the cameras are located near the central axis of the primary body, on a side thereof being opposite a side on which the flexible joint is located relative to the primary body.

20. A device according to claim 19, wherein the spool is mounted on the spool station or on the device.

21. A method of inspecting a pipeline in a single pass including the steps of:
(A) inserting a pipeline-inspecting device into pipeline to be inspected such that a plurality of circumferentially spaced primary contacts extending from the pipeline-inspecting device remains in contact with a 360 degree band of an internal surface of the pipeline
(B) electrically connecting a second contact of the pipeline-inspecting device to the pipeline;
(C) monitoring any changes in voltage, resistance, impedance, conductivity or other electrical property between the primary contacts and the pipeline as the pipeline-inspecting device moves along the pipeline;
(D) on detecting a change in electrical property between the primary contact and the pipeline as a result of a defect, actuating a marking device associated with such primary contact to visually mark the internal surface of the pipeline in the vicinity of the defect detected; and
(E) recording the location of the defect.
